(19) United States
(12) Patent Application Publication
(10) Pub. No.: US 2025/0102488 A1
(43) Pub. Date: Mar. 27, 2025

(54) POLYACRYLAMIDE-CONTAINING INDIRECT NEGATIVE CAVITY WATER PRESSURE MEASUREMENT SYSTEM AND METHOD

(71) Applicant: KADIR HAS ÜNIVERSITESI, Fatih, Istanbul (TR)

(72) Inventors: Asli Senem AÇIKEL, Kirkland, WA (US); Mehmet Önder PEKCAN, Fatih, Istanbul (TR); Bengü Özugur UYSAL, Fatih, Istanbul (TR)

(21) Appl. No.: 18/728,268

(22) PCT Filed: Mar. 24, 2023
(86) PCT No.: PCT/TR2023/050281

§ 371 Date: Jul. 11, 2024

(30) Foreign Application Priority Data
Sep. 13, 2022 (TR) 2022/014178

(51) Int. Cl. G01N 33/24; G01N 1/34; G01N 33/02

(52) U.S. Cl. CPC G01N 33/246; G01N 1/34; G01N 33/02

(57) ABSTRACT

Disclosed are a polyacrylamide-containing indirect negative cavity water pressure measurement system and a polyacrylamide-containing indirect negative cavity water pressure measurement method suitable for use with this system, which simplify the processes of measuring values related to the relative humidity value of any porous medium, such as the negative cavity water pressure of the ground (soil), the water activity values of foods, etc., reducing the measurement costs, increasing the accuracy and precision of the measured values.

### TECHNICAL FIELD

The invention relates to a polyacrylamide-containing indirect negative cavity water pressure measurement system and method for measuring the negative cavity water pressure value in a simple and cost-effective way, and for increasing the measurement precision and accuracy.

In particular, the invention relates to a polyacrylamide-containing indirect negative cavity water pressure measurement system and a polyacrylamide-containing indirect negative cavity water pressure measurement method suitable for use with this system, which simplify the processes of measuring values related to the relative humidity value of any porous medium, such as the negative cavity water pressure of the ground (soil), the water activity values of foods, etc., reducing the measurement costs, increasing the accuracy and precision of the measured values.

### STATE OF THE ART

The water pressure around and between soil grains is called pore water pressure. The pore water pressure (Uw) at any depth is equal to the product of the pyesometric height (free water height) at that point and the unit volume weight of water. The cavity water pressure is hydrostatic and has the same value in all directions. The atmospheric pressure at the water table is also the water pressure level. If the atmospheric pressure is assumed to be zero, the water pressure between particles in a porous medium such as soil, ground, food material, etc. causes positive pore water pressure, while the lack of water causes negative pore water pressure. In this context, since the pore water pressure in an unsaturated porous medium is actually negative, the effective stress is greater than the total stress. This causes the surface tension of the pore water in the voids to create a suction effect on the surrounding particles, resulting in matrix absorption.

Water retention curves (soil-water characteristic curves) are known as the most critical concept of water-insaturated soil mechanics. These curves express the gravimetric or volumetric water content or water saturation rate of the soil as a function of the energy released in the form of suction, water potential, negative pore water pressure or relative humidity and are a measure of water activity in the soil. There are many methods to measure (e.g. filter paper, tensiometer, dew point potentiometer measurement) or calibrate (e.g. vapor balance, osmotic measurement) the negative pore water pressure to obtain water retention curves of porous media. However, none of these methods can effectively meet the basic and necessary criteria such as achieving low cost in the measurement and calibration stages, offering user-friendly operation, providing reliable and accurate results, allowing multiple uses, and having a wide application range. For example, although the filter paper method is known as a low-cost and simple method with a wide measurement range, it is not sufficient in terms of measurement range, accuracy and measurement quality and allows only one-time use.

On the other hand, Polyacrylamide (PAAm) is a highly hydrophilic polymer material that swells with water absorption. The water absorption and swelling of this polymer can be monitored at known relative humidity values and then used to calculate the corresponding negative pore water pressure values of a porous medium such as soil or food. In this way, it is possible to use PAAm as a simple and low-cost indirect measurement method of negative pore water pressure of soils, water activity values of foods and relative humidity of any environment. The structural heterogeneity of gels is significantly influenced by physical properties such as permeability, elasticity and optical properties. Polyacrylamide (PAAm) gels exhibit high permeability due to the inhomogeneous distribution of their cross-links, and therefore, known research in the art focuses on how the swelling stability of PAAm gels and the transport of water molecules within the gels are affected by the inhomogeneity of the polymer network.

Changes in polymer concentration and the ratio of crosslinkers to polymers can alter the network topology and inhomogeneity. The model relating gel composition to swelling ratio, turbidity, elastic modulus and volume fraction is described for PAAm gels. The transition of a gel between the transparent and opaque state, the heterogeneous state, is described as a function of the concentrations of monomer and crosslinker. An in situ photon transmission technique is reported to study the aging of PAAm gels due to multiple swelling, where a continuous decrease of the transmitted light intensity is observed as the PAAm gel swells. The decrease in transmitted light intensity is attributed to the formation of structural inhomogeneities in the gel and swelling, one of the most important processes in gels, is isotropic. For example, in a long cylindrical gel, when the radius increases by 10%, the axial length increases by 10%. By taking into account two opposing effects, osmotic pressure and restraining force, it is possible to understand the elastic and swelling properties of permanent networks. The controlled and isotropic swelling behavior of PAAm allows it to be considered as a reference material, while the optical observation of swelling demonstrates the reliability and accuracy of the light transmission method.

In a patent document numbered GB2504604A in the known state of the art, a test method for determining the negative pore water pressure or matrix absorption of soil is described. The method involves positioning the soil to be measured in contact with a shrinkable microporous material pre-calibrated according to matrix absorption versus moisture content or volume relationship. Thus, the negative pore water pressure or matrix absorption of the soil is measured. This microporous material can be a pure clay, such as bentonite or smectite, whose water retention curve is known or can be determined. This allows for a more reliable measurement than using filter paper. There is no mention in the document of a solution for measuring and calibrating negative cavity water pressure that achieves low cost of measurement and calibration, time-dependent behavior monitoring, user-friendly operation, multiple uses, a wide range of applications and the use of polyacrylamide.

A material testing system for measuring material properties is described in a patent document US20200309711A1 of known art. The system consists of an outer cell containing a first inner volume and an inner cell containing a second volume suitable for the placement of the material sample. The first set of cameras to detect visible light inside the cell and the second set of cameras to detect infrared light and the thermometer to calculate the temperature change of the material and the movement trace between certain points. In the document, there is no mention of a solution for the use of polyacrylamide for negative cavity water pressure measurement to achieve low cost in the measurement and calibration stages, user-friendly use, reliable and accurate results, a wide range of applications and the use of polyacrylamide.

As a result, there is a need for the development of a polyacrylamide-containing indirect negative cavity water pressure measurement system and method that simplifies the processes during the measurement of negative cavity water pressure/water activity/relative humidity value in a porous medium, reduces measurement costs, increases the accuracy and precision of the measured values, offers repeated use and wide application range.

### PURPOSE OF THE INVENTION

The present invention relates to a polyacrylamide-containing indirect negative cavity water pressure measurement system and method that meets the above-mentioned requirements, eliminates possible disadvantages and provides some additional advantages.

The main purpose of the polyacrylamide-containing indirect negative cavity water pressure measurement system and method subject to the invention is to obtain a polyacrylamide-containing indirect negative cavity water pressure measurement system and method that simplifies the processes during the measurement of negative cavity water pressure/water activity/relative humidity value and increases the measurement precision and accuracy.

Another object of the invention is to provide an efficient and user-friendly polyacrylamide-containing indirect negative cavity water pressure measurement system and method that reduces measurement and calibration costs.

Another object of the invention is to provide a functional polyacrylamide-containing indirect negative cavity water pressure measurement system and method, which allows multiple measurements to be made.

Another object of the invention is to provide an effective polyacrylamide-containing indirect negative cavity water pressure measurement system and method, which enables increasing the range and type of applications for different measurement and calibration processes.

Another object of the invention is to provide a precise, qualified and reproducible polyacrylamide-containing indirect negative cavity water pressure measurement system and method that does not come into contact with the material on which the measurement process is applied.

Another object of the invention is to provide an effective polyacrylamide-containing indirect negative cavity water pressure measurement system and method, which enables the consistency and quality of the measurement results to be increased by monitoring the time dependence and dynamic equilibrium change of the measurements.

Another object of the invention is to provide a polyacrylamide-containing indirect negative cavity water pressure measurement system and method for measuring negative cavity water pressure, which enables continuous measurement without the need to intervene in the system, thereby avoiding the prolongation of the equilibration process of the system for the measurement process.

Another object of the invention is to provide a polyacrylamide-containing indirect negative cavity water pressure measurement system and method that enables gas exchange for physical modeling of different atmospheric compositions, and thus, a polyacrylamide-containing indirect negative cavity water pressure measurement system and method suitable for use in different fields such as astrobiology or CO2 underground research, offering a wide range of uses and increasing the variety of measurements.

In order to achieve the above objectives in the most general form, the polyacrylamide-containing indirect negative cavity water pressure measurement system developed with the present invention, which enables the measurement of polyacrylamide, measurement sample and negative cavity water pressure/water activity/relative humidity value, increasing the accuracy and sensitivity of the measured values, allowing repeated measurements to be made time-dependent or independent, comprises at least one transport element that enables the materials placed in it to be transported and isolated from external environmental conditions; at least one positioning element for fixing the position of the transport element and measuring the optical properties of the light transmitted through the transport element.

The polyacrylamide-containing indirect negative cavity water pressure measurement method suitable for use with the polyacrylamide-containing indirect negative cavity water pressure measurement system developed with the present invention comprises performing the calibration process; positioning each of the measurement samples in one of the transport elements in a manner not exceeding half of the volume of the transport element; suspending each of the polyacrylamides in one of the transport elements in such a way that they do not come into contact with the measurement samples; sealing the transport elements so that they are impermeable to gases and liquids and positioning them on the positioning element; passing light in the ultraviolet-visible light range through the transport elements in order to measure the change in the intensity/severity of light passing through the polyacrylamides; measurement of the light intensity/intensity value in the UV-visible light range; dynamic equilibrium of the relative humidity value of the system consisting of polyacrylamide and measurement sample by repeating the measurement procedures and obtaining stabilized measurement values; measurement of the gravimetric water content value of polyacrylamides and measurement samples; calculation of the polyacrylamide negative void water pressure value corresponding to the gravimetric water content value measured in dynamic equilibrium; indirect measurement of the negative void water pressure value corresponding to the measurement sample gravimetric water content value.

The structural and characteristic features and all advantages of the invention will be more clearly understood by means of the figures given below and the detailed description written with references to these figures, and therefore, the evaluation should be made by considering these figures and detailed description.

### FIGURES TO HELP UNDERSTAND THE INVENTION

In order to best understand the structure and advantages of the present invention, it should be evaluated together with the figures described below.

FIG. 1: A cross-sectional view of the inventive polyacrylamide-containing indirect negative cavity water pressure measurement system.

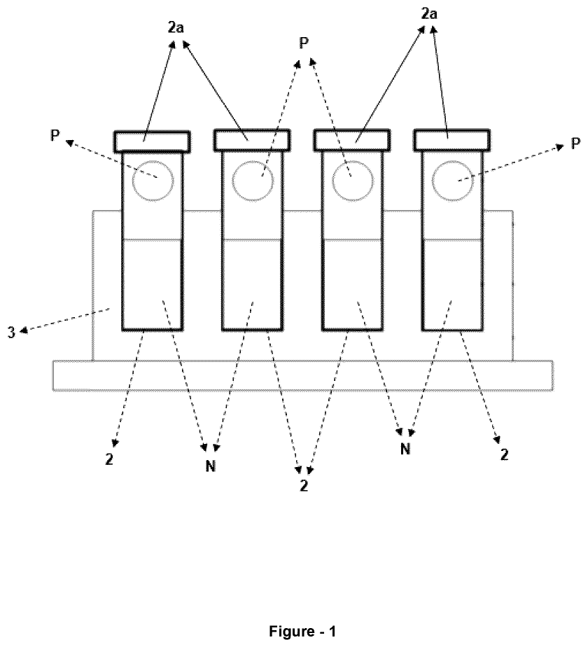

FIG. 2: Another cross-sectional view of the polyacrylamide-containing indirect negative cavity water pressure measurement system subject to the invention.

FIG. 3: A cross-sectional view of the transport element in the polyacrylamide-containing indirect negative cavity water pressure measurement system according to the invention.

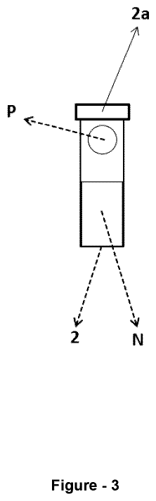

FIG. 4: An algorithm view of the process steps in the inventive polyacrylamide-containing indirect negative cavity water pressure measurement method.

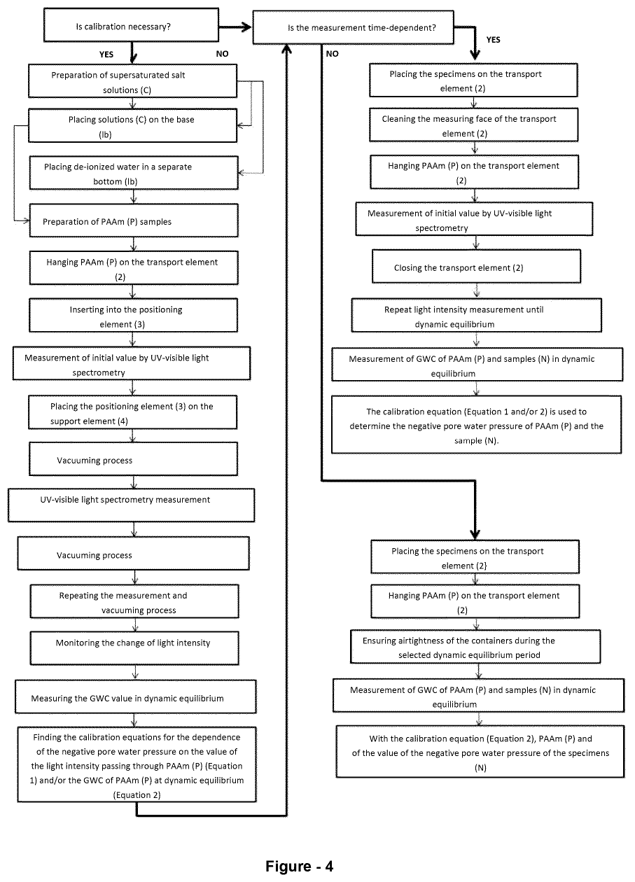

### PART REFERENCES

1. Body element
1a. Hopper
1b. Base
1c. Cover
2. Transport element
2a. Second cover
3. Positioning element
4. Support element
5. Vacuuming element
6. Insulation element
7. Flow control element
8. Transmission element
N. Measurement sample
P. Polyacrylamide

### DETAILED DESCRIPTION OF THE INVENTION

In this detailed description, the preferred embodiments of the inventive polyacrylamide-containing indirect negative cavity water pressure measurement system and method are described only for the purpose of a better understanding of the subject matter.

The polyacrylamide (PAAm) (P) used in the measurement and/or calibration process developed by the present invention, preferably in the form of a disk, the sample (N) on which the measurement process is to be performed and the measurement sample (N) on which the negative pore water pressure/water activity/relative humidity value is measured, which allows the measurement of the negative pore water pressure/water activity/relative humidity value, simplifying the measurement processes and increasing the accuracy and precision of the measured values, time-dependent or independently more than one, a polyacrylamide-containing indirect negative cavity water pressure measurement system allowing repeated measurements, each including at least one second cover (2a) providing gas and liquid sealing, at least one transport element (2), preferably in an optical cuvette structure, preferably suitable for placing at least one polyacrylamide (P) and/or measurement sample (N) inside and preferably suitable for passing light in the ultraviolet-visible light range through it, allowing the materials placed inside to be transported and isolated from external environmental conditions; at least one positioning element (3) in connection with the transport element (2), which enables the position of the transport element (2) to be fixed, preferably including at least one suspension element, which is suitable for passing light in the ultraviolet-visible light range through the transport element (2) and measuring the optical properties of the light passed through the transport element (2), preferably including an ultraviolet-visible light spectrometer, preferably including at least one suspension element that enables the position of the said transport element (2) to be easily changed and fixed.

In an exemplary embodiment of the polyacrylamide-containing indirect negative cavity water pressure measurement system developed with the present invention, after calibration by establishing a water retention approximation curve with gravimetric water content (GWC) values of polyacrylamide (P), preferably using the vapor equilibrium technique (VET), suction measurement and/or calibration techniques, In order to measure the negative pore water pressure values, each of the measurement samples (N) prepared at different water contents is positioned in one of the bearing elements (2) in such a way that it does not exceed half of the volume of the bearing element (2). Each polyacrylamide (P) of the same physical dimensions as the polyacrylamide (P) used in the calibration process is suspended in one of the transport elements (2) in such a way that it does not come into contact with the measurement samples (N). By means of the second caps (2a), the transport elements (2) are sealed gas and liquid-tight and then positioned on the positioning element (3). In order to measure the change in the intensity/intensity of light passing through the polyacrylamides (P) in the different transport elements (2) for time-dependent/dynamic equilibrium monitored measurement, light in the ultraviolet-visible light range is transmitted through the transport elements (2) by means of the positioning element (3) and the light intensity/intensity value in the ultraviolet-visible light range is measured by means of the positioning element (3). In case the measured light intensity values are not stable, the measurement process is repeated until the measured values stabilize and the dynamic equilibrium of the relative humidity value of the system consisting of polyacrylamide (P) and measurement sample (N) in the transport element (2) is achieved by obtaining stabilized measured values. Once the relative humidity reaches dynamic equilibrium in both time-dependent and time-independent measurements, the transport elements (2) are opened and the gravimetric water content value of the polyacrylamides (P) in the transport elements (2) and the measurement samples (N) is measured. Then, using the water retention approximation curve obtained in the calibration process, the polyacrylamide (P) negative void water pressure value corresponding to the gravimetric water content of the polyacrylamide (P) measured at dynamic equilibrium is calculated. An indirect measurement of the negative cavity water pressure value corresponding to the gravimetric water content value of the measuring sample (N), which is equal to the calculated negative cavity water pressure value, is performed.

In another preferred embodiment of the invention, said polyacrylamide-containing indirect negative cavity water pressure measurement system comprises at least one chamber (1a) for maintaining the moisture balance of the materials placed therein, at least one base (1b) providing resistance to chemical materials, and at least one bottom (1a) of the chamber (1a), at least one body element (1), preferably a desiccator, which includes at least one cover (1c), preferably air, which ensures that it is closed so that it does not leak gases and liquids, and which ensures that the materials placed in it are preserved and insulated from external environmental conditions.

In another preferred embodiment of the invention, the polyacrylamide-containing indirect negative cavity water pressure measurement system comprises at least one support member (4) in planar form, preferably having holes/cavities therein, which is positioned in the chamber (1a) and which enables the positioning member (3) and the transport member (2) to be carried at a distance from the base (1b).

In another preferred embodiment of the invention, the polyacrylamide-containing indirect negative cavity water pressure measurement system comprises at least one vacuumizing element (5), preferably a vacuum pump or generator, which is connected to the body element (1), which allows the gas in the body element (1) to be removed to the external environment and a vacuum environment to be created in the chamber (1a).

In a preferred embodiment of the invention, said polyacrylamide-containing indirect negative cavity water pressure measurement system comprises at least one sealing element (6), preferably of o-ring or gasket construction, positioned between the edge of said housing element (1) distal to said base (1b) and said cover (1c), providing gas and liquid tightness.

In another preferred embodiment of the invention, said polyacrylamide-containing indirect negative cavity water pressure measurement system comprises at least one flow control element (7), preferably in the form of a valve, connected with said housing element (1) and enabling control of the vacuuming speed and flow state during the removal of gas from said chamber (1a) to the external environment.

In another preferred embodiment of the invention, said polyacrylamide-containing indirect negative cavity water pressure measurement system comprises at least one transmission element (8), preferably of tubular construction, in connection with said flow control element (7) and said vacuuming element (5), which enables gas transmission during the vacuuming process.

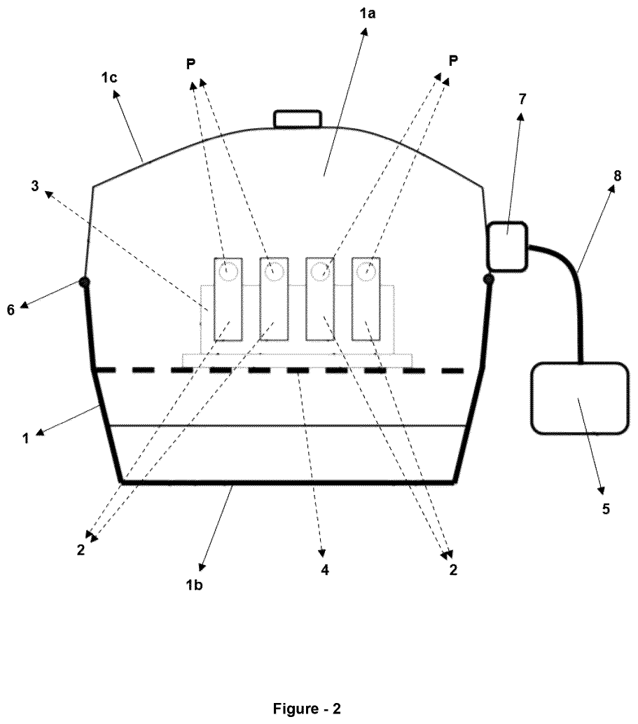

In an exemplary embodiment of the polyacrylamide-containing indirect negative cavity water pressure measurement system developed by the present invention, supersaturated salt solution is placed in the chamber (1a) for calibration using the proposed vapor balance method. Then, the transport elements (2) and the polyacrylamide (P) suspended in the transport elements (2) are positioned on the support element (4) by means of the positioning element (3). The support element (4) ensures that the positioning element (3) and the transport element (2) are positioned in the chamber (la) in such a way that they do not come into contact with the supersaturated salt solution positioned on the base (1b). Polyacrylamide (P) is suspended in the transport element (2). By means of the cover (1c), the body element (1) is closed in such a way that it is impermeable to gas (air) and liquid. By means of the vacuumizing element (5), the gas (air) in the chamber (1a) is taken to the external environment and a vacuum environment is created in the body element (1). After the gas (air) is taken to the external environment, the flow control element (7) is turned off and the vacuum element (5) is closed. By means of the positioning element (3), the optical transmittance value of polyacrylamide (P) is measured by passing light in the ultraviolet-visible light range through the transport elements (2). After each optical transmittance measurement, the positioning element (3) is placed on the support element (4) of the transport element (2) and the polyacrylamide inside. Afterwards, the lid (1c) is closed and the gas (air) in the chamber (1a) is taken to the outside environment by means of the vacuuming element (5) and a vacuum environment is created again in the body element (1). The optical transmittance value of polyacrylamide (P) before the calibration process and the optical transmittance values during the calibration process are monitored and the optical transmittance value measurement and vacuuming process in the calibration setup shown in FIG. 2 are repeated until the measured optical transmittance value reaches dynamic equilibrium. When the measured optical transmittance value reaches dynamic equilibrium, the gravimetric water content (GWC) value of polyacrylamide (P) is measured and this value corresponds to the known relative humidity value or negative void pressure value of the supersaturated salt solution.

The indirect negative void water pressure measurement method suitable for use with the polyacrylamide-containing polyacrylamide-containing indirect negative void water pressure measurement system developed with the present invention, preferably the vapor equilibrium technique (VET), preferably the absorption value measurement depending on the light intensity value in the ultraviolet-visible light range, the negative void water pressure measurement and/or control techniques, and the calibration process by forming the water retention approximation curve with the gravimetric water content (GWC) values of polyacrylamide (P); positioning each of the measurement samples (N) prepared at different water contents for the measurement of negative cavity water pressure values in one of the transport elements (2) in such a way that they do not exceed half of the volume of the transport element (2); hanging each of the polyacrylamides (P) of the same physical dimensions as the polyacrylamide (P) used in the calibration process in one of the transport elements (2) in such a way that they do not come into contact with the measurement samples (N); by means of second covers (2a), the transport elements (2) are sealed gas and liquid-tight and positioned on the positioning element (3); after a waiting time, when the relative humidity has reached dynamic equilibrium, the transport elements (2) are opened and the gravimetric water content of the polyacrylamides (P) in the transport elements (2) and of the measuring samples (N) is measured, preferably using a moisture measuring device or an oven drying method (ASTM D2216-19); calculating the polyacrylamide (P) negative void water pressure value corresponding to the gravimetric water content of the polyacrylamide (P) measured in dynamic equilibrium using the water retention approximation curve obtained in the calibration process; performing an indirect measurement of the negative void water pressure value corresponding to the gravimetric water content value of the measurement sample (N), which is equal to the calculated negative void water pressure value.

In a preferred embodiment of the invention, the polyacrylamide-containing indirect negative pore water pressure measurement method mentioned, the waiting time for the relative humidity to reach dynamic equilibrium is at least 7 days for non-plastic sands and clays with a plasticity index of less than 20, and at least 14 days for materials with a plasticity index greater than 20 (for example, bentonite clay).

In another preferred embodiment of the invention, said polyacrylamide-containing indirect negative cavity water pressure measurement method comprises positioning said positioning element (3) on said positioning element (2) after closing said conveying elements (2) for time-dependent/dynamic equilibrium monitored measurement; passing light in the ultraviolet-visible light range through said positioning element (3) through said conveying elements (2) for measuring a change in the intensity/severity of light passing through the polyacrylamides (P) in different conveying elements (2); measuring, by means of the positioning element (3), the value of the light intensity/intensity in the ultraviolet-visible light range after at least one time value, preferably 1 hour, 8 hours, 1 day, 2 days, 3 days, 4 days, 5 days, 6 days, 7 days and/or 14 days from the switching off of the transport elements (2); if the measured light intensity/intensity values are not stable, repeating the measurement process, preferably on a daily basis, until the measured values stabilize, and achieving dynamic equilibrium of the relative humidity value of the system consisting of the polyacrylamide (P) and the measurement sample (N) in the transport element (2) by obtaining stabilized measured values.

In another preferred embodiment of the invention, said polyacrylamide-containing indirect negative cavity water pressure measurement method comprises the step of preventing contamination of the surface of said transport element (2) with polyacrylamide (P) during positioning of said polyacrylamide (P) in said transport elements (2) and/or cleaning the surfaces of said transport element (2) after positioning in order to increase measurement accuracy and precision in time-dependent/dynamic equilibrium monitoring measurements.

In another preferred embodiment of the invention, said polyacrylamide-containing indirect negative cavity water pressure measurement method, said calibration process comprising positioning at least one supersaturated salt solution, preferably a supersaturated solution of LiBr, LiCl, CH3COOK, K2CO3, NaBr, KI, NaCl, KCl, KNO3 and/or K2SO4 with known relative humidity values at constant temperature, on said base (1b) in said body member (1); positioning of de-ionized water, preferably DI at 100% RH, in another body member (1); preparing a plurality of polyacrylamides (P) having identical physical dimensions; hanging each of the prepared polyacrylamides (P) in one of said transport members (2), preferably three separate polyacrylamides (P) and three separate transport members (2) for each supersaturated salt solution; positioning said transport elements (2) comprising said polyacrylamide (P) on said positioning element (3) and determining a first measurement value by measuring the intensity/severity of light in the ultraviolet-visible light range transmitted through said polyacrylamides (P) by means of the positioning element (3) after at least one time value, preferably 1 hour, 8 hours, 1 day, 2 days, 3 days, 4 days, 5 days, 6 days, 7 days and/or 14 days; after determination of the initial value, positioning said transport elements (2) comprising said polyacrylamide (P) in said reservoir (1a) by means of said positioning element (3) and said support element (4) so as not to contact the supersaturated salt solution on said base (1b); after closing said lid (1c), by means of said vacuuming element (5), vacuuming the gas in said housing element (1), preferably for 30 seconds, and placing said transport elements (2) in a vacuum environment, preferably for 1 hour; preferably replacing the gas, preferably air, in said body member (1) with a gas other than air, such as CO2 or N2; determining a second measurement value by measuring the light intensity/severity values in the ultraviolet-visible light range of the polyacrylamides (P) in the transport elements (2) removed in the vacuum environment; preferably repeating the process of replacing the gas in said body member (1) after each measurement process; after determining the second measurement value, repositioning the transport elements (2) containing polyacrylamide (P) into said chamber (1a) and vacuuming the gas in said body element (1), preferably for 30 seconds; repeating the previous two steps, preferably after a period of 8 hours, and obtaining more than one second measurement value after measurements every day, preferably at the same time, and monitoring the time-dependent changes of the light intensity/severity values passing through the polyacrylamides (P); repeating the measurements until the light intensity/severity value passing through the polyacrylamides (P) stabilizes and determining the dynamic equilibrium of the relative humidity value of the closed system consisting of the supersaturated salt solution in the body element (1) and the polyacrylamides (P) when the measured values stabilize; upon reaching dynamic equilibrium of the relative humidity value, the final gravimetric water content value of the polyacrylamides (P) corresponds to the known relative humidity value of the supersaturated salts on the water retention curve of the polyacrylamides (P); measuring the gravimetric water content value of the polyacrylamides (P) after the relative humidity value reaches dynamic equilibrium, preferably using a moisture measuring device or standard oven drying method (ASTM-D2216 2010); constructing a water retention approximation curve with the known relative humidity values of the supersaturated salts and the corresponding average gravimetric water content value of the polyacrylamides (P) for each value.

The polyacrylamide-containing indirect negative space water pressure measurement system and method developed by the present invention simplifies the process of measuring the negative space water pressure/water activity/relative humidity value using polyacrylamide and ultraviolet-visible light spectrum range, and increases the accuracy and precision of the measured values. In addition, an efficient, functional and effective polyacrylamide-containing indirect negative cavity water pressure measurement system and a polyacrylamide-containing indirect negative cavity water pressure measurement method suitable for use with this system are obtained, which provides an efficient, functional and effective polyacrylamide-containing indirect negative cavity water pressure measurement system that offers a wide range of applications by obtaining measurement suitable for repeated use and use in different technical fields, which reduces measurement costs.

1. A polyacrylamide-containing indirect negative cavity water pressure measurement system that includes polyacrylamide used in the measurement and/or calibration process, a measurement sample on which the measurement process will be performed and the measurement of the negative cavity water pressure/water activity/relative humidity value, simplifying the measurement processes and increasing the accuracy and precision of the measured values, allowing multiple, repeated measurements to be made, time-dependent or independent, the system comprising:
at least one transport element, each of which includes at least one second cover that ensures gas and liquid tightness, is suitable for the placement of at least one polyacrylamide and/or measurement sample inside, and ensures the transport of the materials placed inside and their isolation from external conditions; and
at least one positioning element for fixing the position of said transport element in connection with said transport element.

2. A polyacrylamide-containing indirect negative cavity water pressure measurement system according to claim 1, wherein said polyacrylamide is in disk form.

3. A polyacrylamide-containing indirect negative cavity water pressure measurement system according to claim 1, where the said transport element is suitable for light to pass through it in the ultraviolet-visible light range.

4. A polyacrylamide-containing indirect negative cavity water pressure measurement system according to claim 3, wherein said positioning element is suitable for transmitting light in the ultraviolet-visible light range through said transport element and for measuring the optical properties of the light transmitted through said transport element.

5. A polyacrylamide-containing indirect negative cavity water pressure measurement system according to claim 1, wherein said said gas is in air form.

6. A polyacrylamide-containing indirect negative cavity water pressure measurement system according to claim 3, wherein said transport element has an optical cuvette structure.

7. A polyacrylamide-containing indirect negative cavity water pressure measurement system according to claim 1, wherein the said transport element is suitable for light to pass through it in the ultraviolet-visible light range, and wherein said positioning element contains an ultraviolet-visible light spectrometer.

8. A polyacrylamide-containing indirect negative cavity water pressure measurement system according to claim 1, wherein said positioning element comprises at least one suspension element which enables the position of said transport element to be easily changed and fixed.

9. A polyacrylamide-containing indirect negative cavity water pressure measurement system according to claim 1, comprising at least one chamber to maintain the moisture balance of the materials placed in it, at least one base to provide resistance against chemical materials and at least one cover to ensure that the said chamber is closed in a gas and liquid-tight manner, and at least one body element to ensure that the materials placed in it are kept and isolated from external environmental conditions.

10. A polyacrylamide-containing indirect negative cavity water pressure measurement system according to claim 9, wherein said body element mentioned is the desiccator.

11. A polyacrylamide-containing indirect negative cavity water pressure measurement system according to claim 9, wherein said positioning element is positioned in said receptacle, and at least one support element enables said transport element to be carried at a distance from said base.

12. A polyacrylamide-containing indirect negative cavity water pressure measurement system according to claim 11, wherein said support element has a planar form, with holes/gaps on it.

13. A polyacrylamide-containing indirect negative cavity water pressure measurement system in accordance with claim 9, comprising at least one vacuumizing element connected to said housing element, which allows the gas in said housing element to be removed to the external environment and a vacuum environment to be created in said chamber.

14. A polyacrylamide-containing indirect negative cavity water pressure measurement system according to claim 13, wherein said vacuumizing element is a vacuum pump or generator.

15. A polyacrylamide-containing indirect negative cavity water pressure measurement system according to claim 9, wherein said housing element comprises at least one sealing element, positioned between the edge distal to said base and said cover, providing gas and liquid tightness, preferably with o-ring or gasket construction.

16. A polyacrylamide-containing indirect negative cavity water pressure measurement system according to claim 9, comprising at least one flow control element, preferably in the form of a valve, connected with said housing element and enabling control of the vacuuming speed and flow state during the removal of gas from said chamber to the external environment.

17. A polyacrylamide-containing indirect negative cavity water pressure measurement system according to claim 9, comprising at least one transmission element, preferably of tubular construction, in connection with said flow control element and said vacuuming element, which enables gas transmission during the vacuuming process.

18. A polyacrylamide-containing indirect negative cavity water pressure measurement system that includes polyacrylamide used in a polyacrylamide-containing indirect negative cavity water pressure measurement system according to claim 1, measurement and/or calibration process, the measurement sample on which the measurement process will be performed and the measurement of the negative cavity water pressure/water activity/relative humidity value, simplifying the measurement processes and increasing the accuracy and precision of the measured values, allowing multiple, repeated measurements to be made, time-dependent or independent, comprising the steps of:
calibration of polyacrylamide with gravimetric water content values by constructing a water retention approximation curve;
positioning each of the measuring samples prepared at different water contents for the measurement of negative cavity water pressure values in one of the said conveying elements in such a way that the volume of said conveying element does not exceed half of the volume of said conveying element;
each of the polyacrylamide of the same physical dimensions as the polyacrylamide used in said calibration process is suspended in one of said transport elements in such a way that it does not come into contact with said measurement samples;
by means of said second covers, said transport elements are sealed gas and liquid-tight and positioned on said positioning element;
when the relative humidity reaches dynamic equilibrium, the aforementioned transport elements are opened and the gravimetric water content of the polyacrylamides and the measured samples in the aforementioned transport elements are measured
calculation of the polyacrylamide negative void water pressure value corresponding to the gravimetric water content of polyacrylamide measured in dynamic equilibrium using the water retention approximation curve obtained in the calibration process;
performing indirect measurement of the negative pore water pressure value corresponding to the gravimetric water content value of the measuring sample, which is equal to the calculated negative pore water pressure value.

19. A polyacrylamide-containing indirect negative cavity water pressure measurement method according to claim 18, comprising the step of performing the calibration process by using vapor balance technique, preferably using absorption value measurement based on light intensity value in the ultraviolet-visible light range, negative cavity water pressure measurement and/or control techniques.

20. A polyacrylamide-containing indirect negative cavity water pressure measurement method according to claim 18, wherein the waiting time for the relative humidity to reach dynamic equilibrium is at least 7 days for non-plastic sands and clays with a plasticity index of less than 20 and at least 14 days for materials with a plasticity index greater than 20.

21. A polyacrylamide-containing indirect negative cavity water pressure measurement method according to claim 18, comprising positioning said positioning element on said positioning element after closing said conveying elements for time-dependent/dynamic equilibrium monitored measurement; passing light in the ultraviolet-visible light range through said positioning element through said conveying elements for measuring a change in the intensity/severity of light passing through the polyacrylamides in different conveying elements; measuring, by means of the positioning element, the value of the light intensity/intensity in the ultraviolet-visible light range after at least one time value; if the measured light intensity/intensity values are not stable, repeating the measurement process, until the measured values stabilize, and achieving dynamic equilibrium of the relative humidity value of the system comprising the polyacrylamide and the measurement sample in the transport element by obtaining stabilized measured values.

22. A polyacrylamide-containing indirect negative cavity water pressure measurement method according to claim 21, wherein said time value in the light intensity/severity measurement step is 1 hour, 8 hours, 1 day, 2 days, 3 days, 4 days, 5 days, 6 days, 7 days and/or 14 days in time-dependent measurements.

23. A polyacrylamide-containing indirect negative cavity water pressure measurement method according to claim 21, wherein time-dependent measurements comprise the step of repeating the measurement processes mentioned in the step of monitoring the dynamic balance of the relative humidity value of the system on a daily basis.

24. A polyacrylamide-containing indirect negative cavity water pressure measurement method according to claim 18, comprising the step of gravimetric water content measurement of polyacrylamides and measurement samples using a moisture measurement device or oven drying method.

25. A polyacrylamide-containing indirect negative cavity water pressure measurement method according to claim 21, comprising the step of preventing contamination of the surface of said transport element with polyacrylamide during positioning of said polyacrylamide in said transport elements and/or cleaning the surfaces of said transport element after positioning in order to increase measurement accuracy and precision in time-dependent/dynamic equilibrium monitoring measurements.

26. A polyacrylamide-containing indirect negative cavity water pressure measurement system according to claim 18, wherein said gas is in air form comprises the steps of:
positioning at least one supersaturated salt solution of known relative humidity at constant temperature on said base in said body member;
preparing a plurality of polyacrylamides having identical physical dimensions;
suspending each of the prepared polyacrylamides in one of said transport elements;
positioning said transport elements containing polyacrylamide on said positioning element, and determining a first measurement value by measuring the intensity/severity of light in the ultraviolet-visible light range transmitted through said polyacrylamide by means of said positioning element;
after determination of the initial value, positioning said transport elements comprising said polyacrylamide in said reservoir by means of said positioning element and said support element so as not to contact the supersaturated salt solution on said base;
vacuuming the gas in said body element by means of said vacuuming element after closing said lid and keeping said transport elements in a vacuum environment;
determination of the second measurement value by measuring the light intensity/intensity values in the ultraviolet-visible light range of the polyacrylamides in the transport elements removed in the vacuum environment;
after determination of the second measurement value, repositioning the transport elements containing polyacrylamide into said chamber and vacuuming the gas in said body element;
repeating the previous two steps and obtaining more than one second measurement value after each day's measurements and monitoring the time-dependent changes of the light intensity/severity values passing through the polyacrylamides;
repeating the measurements until the intensity/severity value of the beam passing through the polyacrylamides stabilizes and determining the dynamic equilibrium of the relative humidity value of the closed system comprising the supersaturated salt solution in the body element and the polyacrylamides when the measured values stabilize;
upon reaching dynamic equilibrium of the relative humidity value, the final gravimetric water content value of the polyacrylamides corresponds to the known relative humidity value of the supersaturated salts on the water retention curve of the polyacrylamides;
measurement of the gravimetric water content value of the polyacrylamides after the relative humidity value has reached dynamic equilibrium; and
constructing a water retention approximation curve with the known relative humidity values of the supersaturated salts and the corresponding average gravimetric water content value of the polyacrylamides for each value.

27. A polyacrylamide-containing indirect negative cavity water pressure measurement system according to claim 26, wherein said supersaturated salt solution is a supersaturated solution of LiBr, LiC, CH₃COOK, K₂CO₃, NaBr, KI, NaCl, KCl, KNO₃ and/or K₂SO₄.

28. A polyacrylamide-containing indirect negative cavity water pressure measurement system according to claim 26, comprising the step of positioning de-ionized water with DI at 100% RH into another body element prior to the step of preparing the polyacrylamides.

29. A polyacrylamide-containing indirect negative cavity water pressure measurement system according to claim 26, comprising the step of hanging polyacrylamides, in such a way that at least three separate polyacrylamide and at least three separate transport elements for each supersaturated salt solution.

30. A polyacrylamide-containing indirect negative cavity water pressure measurement method according to claim 26, wherein said time value in the light intensity/severity measurement step is 1 hour, 8 hours, 1 day, 2 days, 3 days, 4 days, 5 days, 6 days, 7 days and/or 14 days.

31. A polyacrylamide-containing indirect negative cavity water pressure measurement system according to claim 26, comprising the step of vacuuming the gas in the body element for 30 seconds.

32. A polyacrylamide-containing indirect negative cavity water pressure measurement system according to claim 26, comprising the step of the transport elements are kept in the vacuum environment of the body element for 1 hour.

33. A polyacrylamide-containing indirect negative cavity water pressure measurement system according to claim 26, comprising the step of replacing the gas which is air in said body member with a gas other than air, such as CO₂ or N₂, before, after or during said vacuum process.

34. A polyacrylamide-containing indirect negative cavity water pressure measurement system according to claim 26, comprising the step of the determination of the second measurement value involves the step of repeating the process of replacing the gas in the aforementioned body element after each measurement operation.

35. A polyacrylamide-containing indirect negative cavity water pressure measurement system according to claim 26, comprising the step of the determination of the second measurement value and the vacuuming of the gas, and repeating the determination of the second measurement value and the vacuuming of the gas 8 hours after the determination of the second measurement value and the vacuuming of the gas.

36. A polyacrylamide-containing indirect negative cavity water pressure measurement system according to claim 35, comprising the step of the second step of determining the measured value after 8 hours is repeated at the same time every day.

37. A polyacrylamide-containing indirect negative cavity water pressure measurement method according to claim 26, comprising the step of measuring the gravimetric water content of polyacrylamides using a moisture measurement device or a standard oven drying method.
